(19) United States
(12) Patent Application Publication
(10) Pub. No.: US 2019/0029885 A1
(43) Pub. Date: Jan. 31, 2019

(54) SUPPORT PADS FOR HEADBAND

(71) Applicant: SERVORE CO., LTD., Gyeonggi-do (KR)

(72) Inventors: Woon Su SEO, Gyeonggi-do (KR); Jeong Min SEO, Gyeonggi-do (KR)

(21) Appl. No.: 16/000,005

(22) Filed: Jun. 5, 2018

(30) Foreign Application Priority Data
Jul. 27, 2017 (KR) 10-2017-0095181

(51) Int. Cl. A61F 9/06; A42B 3/08; A42B 3/14; B23K 9/32

(52) U.S. Cl. CPC A61F 9/06; B23K 9/322; A42B 3/145; A42B 3/085

(57) ABSTRACT

A support pad for a headband disposed on a side of a headband that is worn on a side of the head of a worker to support the back of the worker's head, the support pad including: a coupler formed on a side of the headband; a first pad unit formed at a portion, which corresponds to the right part of the back of the worker's head, to be rotatable left and right; and a second pad unit formed at a portion, which corresponds to the left part of the back of the worker's head, to be rotatable left and right.

### CROSS REFERENCE

The present application claims priority to Korean Patent Application No. 10-2017-0095181, filed 27 Jul. 2017, the entire contents of which is incorporated herein for all purposes by this reference.

### BACKGROUND

The present invention relates to a support pad for a headband. More particularly, the present invention relates to a support pad for a headband that can independently support the right part and the left part of the back of a worker's head, respectively, and can be easily brought in close contact with the head of a worker regardless of the head shape and size of the worker by including a first pad unit and a second pad unit that are coupled to sides of the headband to be able to freely rotate.

A welding is a metal process that joins two or more solid metals together. Generally, the welding requires a high energy source. A high pressure electricity or a high pressure and explosive gas such as oxygen, acetylene, or argon etc. are used as the high energy source. The maleficence and hazards according to the welding are caused by welding fumes (metal components contained in the fumes), harmful rays, noises, and high temperature environments etc. that are generated in welding work.

Here, the welding fume is a fine particle generated by a cooling of a material evaporated by a heat during welding and is generated through a diffusion of molten metal vapors due to high-temperature arc heat. In the shielded metal arc welding, the relationship between the amount of fume generation and the welding currents shows a tendency in that the amount of generation increases as a current, a voltage, and a diameter of welding rod increase.

In addition, strong ultraviolet rays and infrared rays, which are harmful rays generated, are fatal to human eyes. Therefore, during welding thereof, welding helmets with shading plates are used to protect workers from the harmful rays and the welding fumes.

FIG. 1A is a perspective view showing a conventional welding mask and FIG. 1B is a perspective view showing a conventional support pad for a welding mask headband.

As shown in FIG. 1A and FIG. 1B, the conventional welding mask includes a mask body (110) disposed in front of a face of a worker and having a light-shading plate (111) formed on one side thereof corresponding to an eye of the worker and a headband (120) connected to a rear side of the main body (110) and having one side supported on the head of the worker.

In the conventional welding mask, the headband (120) is worn to cover the head of the worker, so that the mask body (110) is disposed at a position corresponding to the face of the worker. At this time, the worker confirms the working position by deattaching the mask body (110) for a while during confirmation of the working position and performs the work through the shading plate (111) formed in the mask body (110) during working thereof.

At this time, the conventional welding mask (100) can be further coupled with support pads (130) at a position corresponding to the occipital region of the worker located on one side of the headband (120), so that the wearing state of the mask body 110 can be maintained.

As shown in FIG. 1B, the conventional support pad 130 for welding mask headband is formed in a substantially rectangular shape and is fixedly coupled to a position corresponding to the occipital region of the worker located on one side of the headband (120). However, when the mask body (110) is used for common use, since it is difficult to be closely supported by various head parts, its supporting force is low. In addition, when the mask body (110) is moved by an external force, there is a problem in that the supporting position thereof is displaced and the supporting force thereof is remarkably lowered.

### SUMMARY OF THE INVENTION

The present invention has been made in consideration of the problems described above and an object of the present invention is to provide a support pad for a headband that can independently support the right part and the left part of the back of a worker's head, respectively, and can be easily brought in close contact with the head of worker regardless of the head shape and size of the worker by including a first pad unit and a second pad unit that are coupled to sides of the headband to be able to freely rotate.

According to an aspect of the invention to achieve the object described above, there is provided a support pad for a headband that is disposed on a side of a headband that is worn on a side of the head of a worker to support the back of the worker's head, the support pad including: a coupler formed on a side of the headband; a first pad unit formed at a portion, which corresponds to the right part of the back of the worker's head, to be rotatable left and right; and a second pad unit formed at a portion, which corresponds to the left part of the back of the worker's head, to be rotatable left and right, wherein the first pad unit and the second pad unit independently support the right part and the left part of the back of the worker's head, respectively, and turn left and right in accordance with movement of the worker.

Preferably, the first pad unit and the second pad unit each have a support body; a shaft vertically formed behind the pad body and coupled to a side of the coupler to be rotatable left and right; and a pad part coupled to a side of the support body to be brought in close contact with a side of the back of the worker's head.

Preferably, the coupler has a first coupling protrusion formed at a side corresponding to the right part of the back of the worker's head, having a first slit formed on a side, and detachably coupled to the shaft of the first pad unit through the first slit; and a second coupling protrusion formed at a side corresponding to the second part of the back of the worker's head, having a second slit formed on a side, and detachably coupled to the shaft of the second pad unit through the second slit, and the coupler is integrally formed with the headband at on a side of the headband.

### BRIEF DESCRIPTION OF THE DRAWINGS

The above and other objects, features and advantages of the present invention will be more apparent from the following detailed description taken in confluence with the accompanying drawings, in which:

FIG. 1A is a perspective view showing a conventional welding mask;

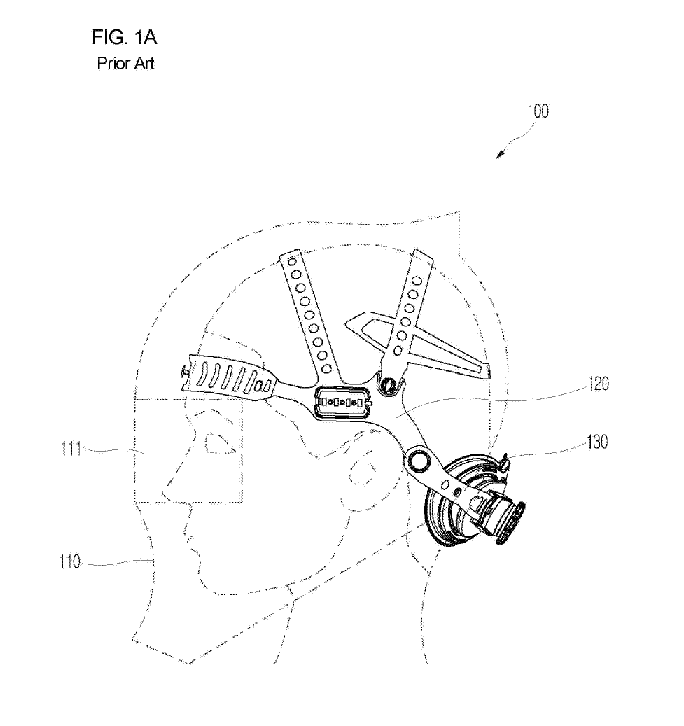

FIG. 1B is a perspective view showing a conventional support pad for a welding mask headband;

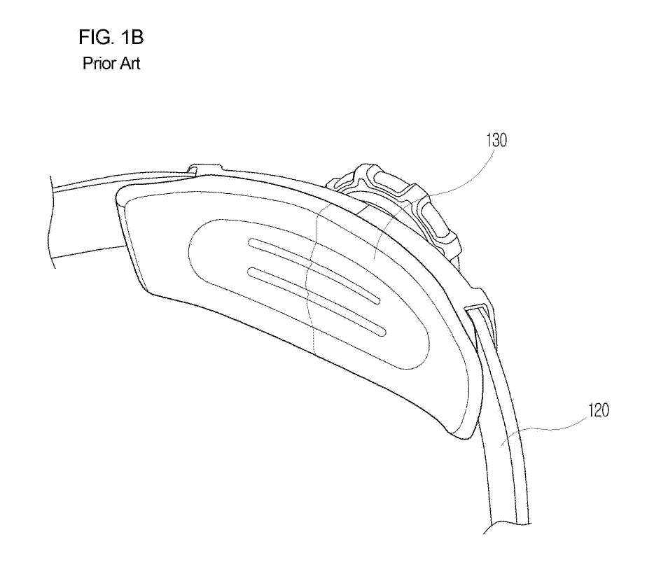

FIG. 2 is a perspective view of a welding mask to which a support pad for a headband according to an embodiment of the present invention is applied;

FIG. 3 is an exploded perspective view of a support pad for a headband according to an embodiment of the present invention;

FIG. 4 is a plan view of a support pad for a headband according to an embodiment of the present invention; and

FIG. 5 is a view showing a state in that a support pad for a headband according to an embodiment of the present invention is closely supported by a worker.

### DETAILED DESCRIPTION OF THE PREFERRED EMBODIMENT

Hereinafter, a preferred embodiment according to the present invention will be described in detail with reference to the accompanying drawings. Since the description of the present invention is a mere embodiment for structural and functional description, it must not be interpreted that the scope of the present invention is limited by the embodiments described in the text. That is, since the embodiments can be variously changed and have various forms, it should be understood that the scope of the invention includes the equivalents for realizing the technical concept. Also, since the specific embodiments do not include all objects and effects presented by the present invention, the scope of the present invention is not limited by them. This invention can be implemented in many different forms without departing from technical aspects or main features.

Hereinafter, an exemplary embodiment of the present invention is described in detail with reference to the accompanying drawings.

A support pad for a headband of the present invention can be applied to all products having a headband such as a welding mask, a respirator, and a safety helmet, and it is exemplified in the embodiment that the support pad of the present invention is applied to a headband for welding masks for the convenience of description.

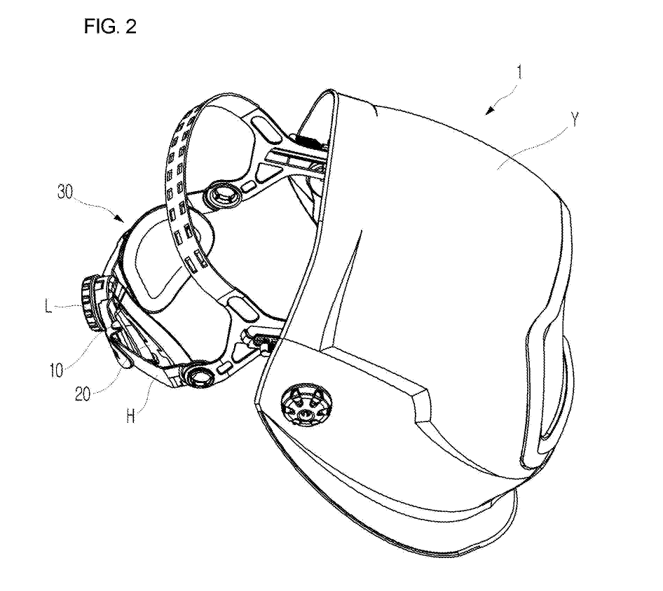

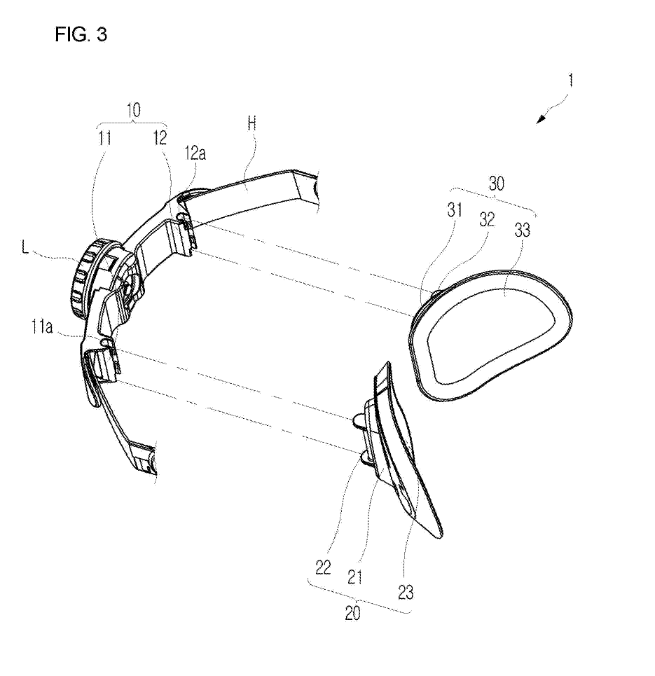

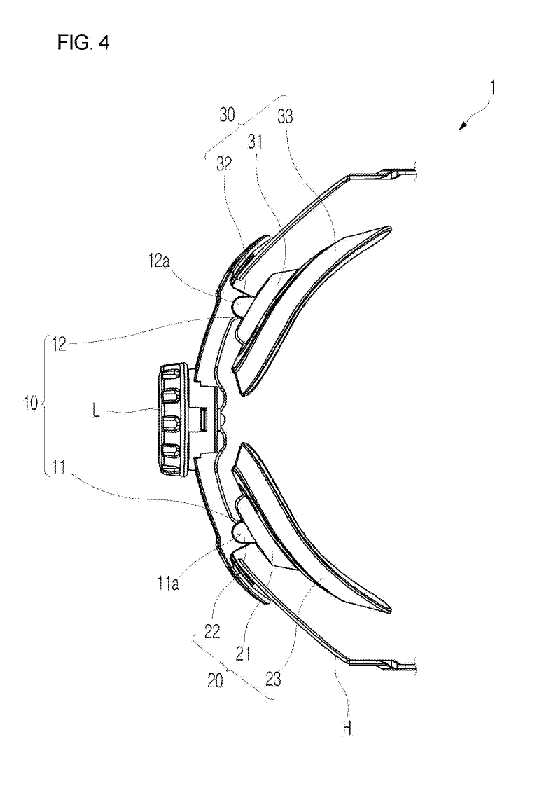

FIG. 2 is a perspective view of a welding mask to which a support pad for a headband according to an embodiment of the present invention is applied, FIG. 3 is an exploded perspective view of the support pad for a headband according to an embodiment of the present invention, and FIG. 4 is a plan view of the support pad for a headband according to an embodiment of the present invention.

As shown in FIGS. 2 to 4, a support pad (1) for a headband according to an embodiment of the present invention includes a coupler (10), a first pad unit (20), and a second pad unit (30).

The coupler (10) is integrated with a latch body (L) allowing for adjustment of the length of a welding mask headband (H) on a side of the headband (H) and has a first coupling protrusion (11) formed at a side, which corresponds to the right part of the back of a worker's head, of the latch body (L) and a second coupling protrusion (12) formed at a side, which corresponds to the left part of the back of a worker's head.

The first coupling protrusion (11) and the second coupling protrusion (12) respectively have a first slit (11a) and a second slit (12a) that are vertically formed on sides of the coupling protrusions, and locking steps are formed at the ends of the slits.

That is, the first slit (11a) and the second slit (12a) of first coupling protrusion (11) and the second coupling protrusion (12) are respectively fitted on a first shaft (22) and a second shaft (32) of the first pad unit (20) and the second pad unit (30) so that the first pad unit (20) and the second pad unit (30) are held on a side of the headband (H).

The first pad unit (20) includes a first support body (21) formed in a substantially rectangular shape, the first shaft (22) vertically formed in a rod shape behind the first support body (21) and coupled to the first coupling protrusion (11) through the first slit (11a) to be rotatable left and right, and a first pad part (23) coupled to a side of the first support body (21) to come in close contact with the right part of the back of a worker's head. Accordingly, the first pad unit (20) supports the right part of the back of a worker's head in close contact with it, so the worker can firmly wear a mask.

The second pad unit (30) includes a second support body (31) formed in a substantially rectangular shape, the second shaft (32) vertically formed in a rod shape behind the second support body (31) and coupled to the second coupling protrusion (12) through the second shaft (32) to be rotatable left and right, and a second pad part (33) coupled to a side of the second support body (31) to come in close contact with the left part of the back of a worker's head. Accordingly, the second pad unit (30) supports the left part of the back of a worker's head in close contact with it, so the worker can firmly wear a mask.

According to the present invention, since the first pad unit (20) and the second pad unit (30) are detachably coupled to the first coupling protrusion (11) and the second coupling protrusion (12), respectively, they can be easily replaced with other pad units, so maintenance is very easy. Further, the pad units independently support the left and right parts of the back of a worker's head in close contact with them, so they can provide improved fit to the worker and can keep in close contact with the back of the worker's head by turning left and right in accordance with movement of the worker.

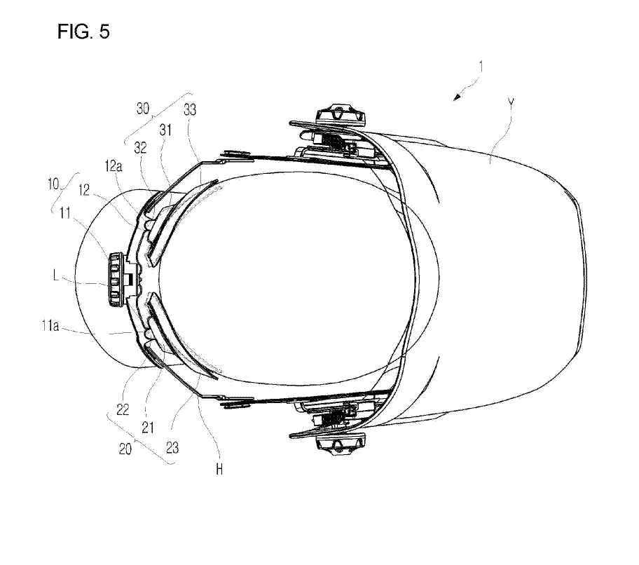

FIG. 5 is a view showing a state in which the support pad for a headband according to an embodiment of the present invention is in close contact with a worker.

The operation of the support pad (1) for a headband according to an embodiment of the present invention having the configuration is described hereafter with reference to FIG. 5.

First, a worker puts the headband (H) of a welding mask on a part of his/her head with the welding face (Y) of the welding mask positioned to correspond to his/her face, in which the first pad unit (20) and the second pad unit (30) at the positions corresponding to the latch body (L) of the headband (H) respectively support the right part and the left part of the back of the worker's head.

The first pad unit (20) and the second pad unit (30), as shown in FIG. 5, are brought in contact with the back of the worker's head by independently and freely turning to fit the right part and the left part of the back of the worker's head. Further, when the worker tightens the headband to fit his/her head shape, the pad units are further brought in contact with the back of the worker's head.

Since the first pad unit (20) and the second pad unit (30) according to the embodiment independently support the right part and the left part of the back of the worker's head, they can be smoothly brought in contact with left-right asymmetric heads, so they can be easily brought in contact with the heads of workers regardless of the head shapes and sizes of workers.

According to the present invention, there are effect in that it can independently support the right part and the left part of the back of a worker's head, respectively, and can be easily brought in close contact with the head of worker regardless of the head shape and size of the worker by including a first pad unit and a second pad unit that are coupled to sides of the headband to be able to freely rotate.

While the present invention has been described with respect to the specific embodiments, it will be apparent to those skilled in the art that various changes and modifications may be made without departing from the spirit and scope of the invention as defined in the following claims.

What is claimed is:

1. A support pad for a headband that is disposed on a side of a headband that is worn on a side of the head of a worker to support the back of the worker's head, the support pad comprising:
a coupler formed on a side of the headband;
a first pad unit formed at a portion, which corresponds to the right part of the back of the worker's head, to be rotatable left and right; and
a second pad unit formed at a portion, which corresponds to the left part of the back of the worker's head, to be rotatable left and right,
wherein the first pad unit and the second pad unit independently support the right part and the left part of the back of the worker's head, respectively, and turn left and right in accordance with movement of the worker.

2. The support pad according to claim 1, wherein the first pad unit and the second pad unit each have:
a support body;
a shaft vertically formed behind the pad body and coupled to a side of the coupler to be rotatable left and right; and
a pad part coupled to a side of the support body to be brought in close contact with a side of the back of the worker's head.

3. The support pad according to claim 2, wherein the coupler has:
a first coupling protrusion formed at a side corresponding to the right part of the back of the worker's head, having a first slit formed on a side, and detachably coupled to the shaft of the first pad unit through the first slit; and
a second coupling protrusion formed at a side corresponding to the second part of the back of the worker's head, having a second slit formed on a side, and detachably coupled to the shaft of the second pad unit through the second slit, and
the coupler is integrally formed with the headband at on a side of the headband.
